(19) United States
(12) Patent Application Publication
(10) Pub. No.: US 2014/0051730 A1
(43) Pub. Date: Feb. 20, 2014

(54) THERAPEUTIC USE OF DIMIRACETAM TO PREVENT THE HAND AND FOOT SYNDROME CAUSED BY SORAFENIB

(71) Applicants: Ruggero G. Fariello, Lugano (CH); Carlo Farina, Vasolda (CO) (IT); Carla Ghelardini, Candeglia (Pistoia) (IT)

(72) Inventors: Ruggero G. Fariello, Lugano (CH); Carlo Farina, Vasolda (CO) (IT); Carla Ghelardini, Candeglia (Pistoia) (IT)

(73) Assignee: NEUROTUNE AG, Schlieren (CH)

(21) Appl. No.: 14/004,389

(22) PCT Filed: Oct. 25, 2011
(86) PCT No.: PCT/CH11/00259

§ 371 Date: Sep. 10, 2013

(30) Foreign Application Priority Data
Oct. 29, 2010 (CH) 1815/10

(51) Int. Cl. A61K 31/4188; A61K 45/06; A61K 31/44

(52) U.S. Cl. CPC A61K 31/4188; A61K 31/44; A61K 45/06
USPC 514/350; 548/302.7; 514/387

(57) ABSTRACT

Dimiracetam is a suitable drug for treating and preventing allodynia, in particular allodynia of the hands and feet, that is or may be induced by antitumoral chemotherapeutic treatment with sorafenib.

### CROSS REFERENCES TO RELATED APPLICATIONS

This application claims the priority of Swiss patent application no. 1815/10, filed Oct. 29, 2010 the disclosure of which is incorporated herein by reference in its entirety.

### TECHNICAL FIELD

The present invention relates to the field of pharmacological treatment of chronic pain in connection with cancer treatment.

### BACKGROUND ART

Chronic neuropathic pain (NeP) is frequently observed in patients receiving antitumoral chemotherapy (Kennedy, J. Med. Chem. 50: 2547-2556, 2007). The use of these therapies for a variety of cancers is significantly limited by the development of a painful peripheral neuropathy. Treatment with antineoplastic drugs in the oncologic patient frequently causes the development of peripheral neuropathy consisting of mechanical and thermal allodynia, spontaneous burning pain, tingling and numbness, allodynic sensation in the hands and/or feet and a chronic foot/leg, hand/arm numbness and pain (Cersosimo, Ann. Pharmacother. 39:128-35, 2004). Although chemotherapeutic agents may differ in the mechanisms by which they induce neurotoxicity, they all produce painful effects often responsible for the therapy interruption. Most “classical” analgesic drugs are generally of little help in relieving this type of NeP which, therefore, remains an important unmet medical need. It is worthy of note that there is currently no effective treatment to prevent or reverse this painful condition. Conceivably, pain induced interruption of the planned chemotherapy may diminish chances of responding to such treatment.

Awada et al. (Br J Cancer. 92:1855-1856, 2005), Cicek et al, (Clin Drug Investig. 28 :803-807, 2008) and Herrmann et al., (J Cancer Res. Clin. Oncol. 2009 135:61-67) reported the development of pain on hands and/or feet of patients three weeks after treatment with the new chemotherapy agent sorafenib.

The inventors have now found that, indeed, administration of sorafenib to rats causes a very strong cold allodynia. They also found that this cold allodynia can be prevented by co-administration of dimiracetam, irrespective of whether the allodinya is acutely or chronically induced.

### DISCLOSURE OF THE INVENTION

Hence, it is a general object of the invention to provide an effective substance and a pharmaceutical composition and a method for the treatment of sorafenib induced allodynia, in particular allodynia in hands and/or feet.

Dimiracetam (2,5-dioxohexahydro-1H-pyrrolo[1,2-a]imidazole) is a bicyclic pyrrolidinonic derivative of formula (I)

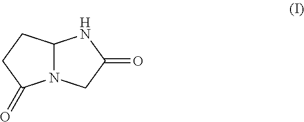

A first object of the present invention is dimiracetam, or a pharmaceutically acceptable solvate thereof, for use in the treatment or prevention of sorafenib induced allodynia, in particular allodynia of hands and/or feet, or the use of dimiracetam, or a pharmaceutically acceptable solvate thereof, in the manufacture of a medicament useful for treating and/or preventing sorafenib induced allodynia, in particular allodynia of hands and/or feet.

A further object of the present invention is a method for treating and/or preventing sorafenib induced allodynia, in particular allodynia of hands and/or feet, consisting in the administration of a pharmaceutically effective amount of dimiracetam to a patient in need thereof.

Also objects of the present invention are pharmaceutical compositions comprising dimiracetam and sorafenib and articles of manufacture comprising a pharmaceutical composition, a written description and instructions of use (e.g. a package insert), and a container.

Dimiracetam is a chiral compound. For the scope of the present invention, the term “dimiracetam” identifies the isolated (R) or (S) enantiomers of dimiracetam, or mixtures thereof in which the two enantiomers are present in equal or different amounts. It is therefore intended that the compound for use, the use, the method and the pharmaceutical compositions which are the object of the present invention enclose any mixtures of the (R) and (S) enantiomers or the single enantiomers of dimiracetam. Also, unless specified, the term dimiracetam as used below also includes its pharmaceutically acceptable solvates (including hydrates), wherein the not solvated dimiracetam usually is preferred.

According to the present invention, dimiracetam may be administered as such or in combination with sorafenib or a sorafenib comprising antitumoral chemotherapeutic combination.

Below, sorafenib and sorafenib comprising antitumoral chemotherapeutic combinations are also referred to as active antitumoral chemotherapeutic principles or merely chemotherapeutic principles. A presently preferred active antitumoral chemotherapeutic principle is sorafenib alone.

By means of the present invention it is possible to treat effectively and with high safety the sorafenib induced pains.

Dimiracetam may be administered prophylactically starting before the administration of the antitumoral chemotherapeutic principle, simultaneously with the antitumoral chemotherapeutic principle or at a later stage of the antitumoral chemotherapeutic therapy. Dimiracetam and sorafenib may both be administered in the same or different fixed intervally, e.g. on a daily basis simultaneous, consecutively or at different times, or one twice a day and the other once all two days etc.

Sorafenib may be used in any amounts suitable for the needed treatment. Usually sorafenib is orally administered in amounts of 5 to 10 mg per kg body-weight. Recommended are daily oral dosages of 400 mg.

The administration dosage of dimiracetam can be varied according to the severity of the neurotoxicity to be treated, the route of administration, the type of chemotherapeutic principle in use (i.e. sorafenib alone or in combination with other antitumoral chemotherapeutic drugs), the patient condition, etc.

In the scope of the invention, i.e. in the treatment addressed, the effect of dimiracetam is exerted in a range of oral dosages between about 1 and about 100 mg/kg, preferably between about 8 and about 70 mg/kg, much preferred between about 15 and about 65 mg/kg. The effect may be achieved also by routes of administration different from the oral route, i.e. intramuscular or intravenous or rectally. In these cases dimiracetam is usually administered in amounts which allow to obtain haematic levels of about 50 to about 750 micromolar, such as about 150 to about 500 micromolar. These blood levels are similar to the blood levels usually (but not necessarily with every patient) induced by about 50 to about 70 mg/kg daily oral administration of dimiracetam. Reference values useful for intramuscular administrations range from about 5 to about 25 mg/kg, preferably about 5 to about 20 mg/kg; reference values useful for intravenous administrations range from about 2 to about 20 mg/kg, preferably about 5 to about 20 mg/kg; and reference values useful for rectal administrations range from about 8 to about 65 mg/kg, preferably about 15 to about 35 mg/kg.

As documented in the experimental part, only for dimiracetam a significant effect was found in rats treated with sorafenib and bearing the symptoms of cold allodynia, whereas no significant effect was found for the used reference drugs gabapentin and duloxetine.

The present invention finds thus substantial utility in improving the practical applicability of sorafenib antitumoral chemotherapy, in that it reduces the associated allodynia side effects and improves patient's acceptability of the anticancer treatment.

Dimiracetam or a solvate thereof may be administered in conjunction with the chemotherapeutic principle: this can be effected either by separate but simultaneous administrations of the active principles, or by administration of a single dosage unit comprising an admixture or combination of the active principles.

Dimiracetam or a solvate thereof can also be used in advance to an antitumoral chemotherapeutic treatment, so as to prevent the development of allodynia. In this case the treatment with dimiracetam is started before the chemotherapeutic treatment and possibly continues jointly therewith.

Dimiracetam or a solvate thereof is also useful in treating possible allodynia symptoms developing after conclusion of the treatment with antitumoral chemotherapeutic drugs; in this case the treatment with dimiracetam is started (or continued) after conclusion of the anticancer treatment.

Dimiracetam or a solvate thereof was also found not to develop tolerance, which is of, fundamental importance in cases, where the therapeutic intervention needs being continued over a long period of time.

A further object of the present invention is the use of a combination of dimiracetam or a solvate thereof, with sorafenib and optionally one or more further antitumoral chemotherapeutic agents, in the manufacture of a medicament for the treatment of cancer, said treatment being advantageously free from allodynia side-effects.

The invention therefore also encompasses pharmaceutical compositions comprising dimiracetam useful for the above mentioned treatment. These compositions contain an amount of dimiracetam (as a first active principle) and optionally also sorafenib or a sorafenib comprising antitumoral chemotherapeutic composition (as a second active principle), both active principles in effective amounts.

Dimiracetam or the solvates thereof or dimiracetam comprising compositions of effective substances (e.g. compositions of active principles as indicated above) may be pharmaceutically formulated according to known methodologies. The various pharmaceutical compositions may be selected according to the needs of the treatment. The pharmaceutical compositions of the invention can be adapted for the various administration routes, and be provided for example in the form of injectable solutions, solutions for infusion, solutions for inhalation, suspensions, emulsions, syrups, elixirs, drops, suppositories, tablets, coated tablets, hard or soft capsules, microcapsules, granules, microgranules, pellets, dispersible powders, lotions, creams, ointments, medicated patches, etc. These compositions also include sustained release formulations.

The amounts of dimiracetam to be administered, expressed in mg/kg, are those cited above. The pharmaceutical compositions addressed above may be present as at least one usually more dosage units useful to administer the above mentioned dosages. Typically such dosage units contain dimiracetam in amounts suitable for daily administration of from 50 to 5000 mg in case of oral compositions, preferably in amounts of 400 to 3500 mg, more preferred 800 to 3200 mg; from 250 to 1200 mg in case of intramuscular compositions, preferably from 400 to 1000 mg; from 100 to 1000 mg in case of intravenous compositions, preferably from 400 to 1000 mg; and from 400 to 3500 mg for rectal compositions (suppositories), preferably at most 3200 mg and much preferred from 800 to 1600 mg. These dosages are calculate for 50 kg body weight as suggested by the US Food and Drug Administration (FDA). In case of daily dosages in the upper ranges, it may be advantageous (or even necessary, e.g. for solid oral dosage units) to provide the daily dosage in several dosage units (e.g. 3000 mg divided into 6 to 10 dosage units for administration one to—preferably—several times a day,

A list comparing the amounts dependent on the administration route used is found in Table 1:

TABLE 1

| Route | Daily dose (mg) | Daily dose (mg/kg) |
|---|---|---|
| Oral | 50-5000 | 1-100 |
| | 400-3500 (preferred) | 8-70 (preferred) |
| | 800-3200 (more preferred) | 15-65 (more preferred) |
| Intravenous | 100-1000 | 2-20 |
| | 400-1000 (preferred) | 5-20 (preferred) |
| Intra- | 250-1200 | 5-25 |
| muscular | 400-1000 (preferred) | 8-20 (preferred) |
| Intrarectal | 400-3500 | 8-70 |
| (suppositories) | 500-3200 (preferred) | 10-65 (preferred) |
| | 800-1600 (more preferred) | 15-35 (more preferred) |

The daily dosages in mg are calculated for an average person of about 50 kg and may be adapted to other body weights. In addition, two or more administration routes may be combined with respectively reduced amounts per route.

The dimiracetam may be formulated alone, e.g. for the treatment of sorafenib induced pain in case that the antitumoral chemotherapeutic treatment with sorafenib or a sorafenib comprising active chemotherapeutic principle is started subsequent to a prophylactic treatment with dimiracetam, or it may be co-formulated with sorafenib or a sorafenib comprising active chemotherapeutic principle in the same administration form, i.e. in the same tablet etc. or in different administration forms allowing the optimized administration of both medications. In the last mentioned kind of formulation, one kind of tablets may comprise the dimiracetam and a second kind of tablets may comprise the active antitumoral chemotherapeutic principle, or one kind of tablets may comprise a mixture of dimiracetam and the active antitumoral chemotherapeutic principle and a second kind of tablets either dimiracetam or active antitumoral chemotherapeutic principle. By such different forms of confectioning the treatment may be optimized.

Suitable pharmaceutical compositions can be prepared by mixing and they can be suitably adapted for oral or parenteral administration, and as such, can be administered in the form of oral preparations, e.g. solid preparations like tablets, capsules, powders, granules, pellets, or liquid preparations like solutions, emulsions or suspensions; or as liquid solutions or suspensions for injection or infusion; or as suppositories.

The excipients optionally and preferably present in these compositions are those commonly used in pharmaceutical technology; they can be used in the manner and quantity commonly known to the expert of the art.

Tablets and capsules for oral administration are usually supplied in dosage units and may contain conventional excipients such as binders, fillers, diluents, tableting agents, lubricants, detergents, disintegrants, colorants, flavors and wetting agents. Tablets may be coated in accordance to methods well known in the art.

Suitable fillers include for example cellulose, mannitol, lactose and similar agents. Suitable disintegrants include starch, polyvinylpyrrolidone and starch derivatives such as sodium starch glycolate. Suitable lubricants include, for example, magnesium stearate. Suitable wetting agents include for example sodium lauryl sulfate.

These solid oral compositions can be prepared with conventional mixing, filling or tableting methods. The mixing operations can be repeated to disperse the active agent in compositions containing large quantities of fillers. These operations are conventional.

The oral liquid compositions can be provided in the form of, for example, aqueous or oily suspensions, solutions, emulsions, syrups or elixirs or in the form of a dry product to be reconstituted with water or with a suitable liquid carrier at the time of use. The liquid compositions can contain conventional additives such as suspending agents, for example sorbitol, syrup, methylcellulose, gelatin, hydroxyethylcellulose, carboxymethylcellulose, aluminium stearate gel or hydrogenated edible fats, emulsifying agents, for example lecithin, sorbitan monooleate, or acacia; non aqueous carriers (which can include edible oil) for example almond oil, fractionated coconut oil, oily esters such as glycerin esters, propylene glycol or ethyl alcohol; preservatives, for example methyl or propyl p-hydroxybenzoate or sorbic acid and if desired, conventional flavours or colorants.

Oral formulations also include conventional sustained release formulations, such as tablets or granules with enteric coating.

For parenteral administration, fluid dosage units can be prepared containing the active compounds and a sterile carrier. The active compounds, depending on the carrier and concentration, can be suspended or dissolved. The parenteral solutions are normally prepared by dissolving the compound in a carrier and sterilizing by filtration, before filling suitable vials or ampoules and sealing. Adjuvants such as local anaesthetics, preservatives and buffering agents can be advantageously dissolved in the carrier. In order to increase stability, the composition can be frozen after filling the vial and the water removed under vacuum. The parenteral suspensions are prepared essentially in the same way, with the difference that the active compounds can be suspended rather than dissolved in the carrier, and can be sterilized by exposure to ethylene oxide prior to being suspended in the sterile carrier. A surfactant or humectant can be advantageously included to facilitate uniform distribution of the compound of the invention.

A further method of administration is via a topic treatment. Topic formulations may contain for example ointments, creams, lotions, gels, solutions, pastes and/or may contain liposomes, micelles and/or microspheres.

A further method of administration is transdermal delivery. Typical transdermal formulations include conventional aqueous and non-aqueous vectors, such as creams, oil, lotions or pastes or may be in the form of membranes or medicated patches.

Also possible is administration via suppositories, especially rectal suppositories. A typical formulation of suppositories comprises one or more active substances, e.g. dimiracetam or an active principle comprising dimiracetam and an active antitumoral chemotherapeutic principle and a binding and/or lubricating agent, for example polymeric glycols, gelatine, cocoa butter, or other low- melting waxes or vegetable fats.

As is the common practice, the compositions are usually accompanied by written or printed instructions for use in the treatment concerned, e.g. a package insert. Thus, also an article of manufacture comprising a composition of the present invention, preferably in dosage units of identical or different composition as outlined above, a written description and administration instruction also known as package insert) and a container or package is encompassed by the present invention.

### MODES FOR CARRYING OUT THE INVENTION

Examples of the present invention are provided in what follows. These Examples are purely for illustrative and non-limiting purposes.

### EXPERIMENTAL DETAILS

Male Sprague Dowley adult rats (200-220 g—aged 7 weeks) were used. Groups of 4 rats were housed in 26 by 41 cm cages that were placed in the experimental room 24 h before the test for acclimatization. The animals were fed a standard laboratory diet and tap water ad libitum and kept at 23±1° C. with a 12 h light/dark cycle, light on at 7 a.m. All experiments were carried out in accordance with the European Communities Council Directive of 24 Nov. 1986 (86/609/EEC) for experimental animal care. All efforts were made to minimize the number of animals used and their suffering. The compounds used for comparative purposes were employed at the doses that were reported in the literature as maximally active in other animal models of neuropathic pain (Iyengar S. et al., Efficacy of Duloxetine, a Potent and Balanced Serotonin-Norepinephrine Reuptake Inhibitor in Persistent Pain Models in Rats, The Journal of Pharmacology and Experimental Therapeutics, JPET 311:575-584, 2004, and Field M. J. et al., Gabapentin and the Neurokininl Receptor Antagonist CI-1021 Act Synergistically in Two Rat Models of Neuropathic Pain, JPET 303:730-735, 2002).

Blood levels were determined in healthy volunteers during Phase I studies.

Drugs

Dimiracetam, duloxetine and gabapentin were dissolved in water. Drug concentrations were adjusted to a volume of 10 ml/kg for dosing by oral administration or i.p. injection. Sorafenib was dissolved in a vehicle composed by 1% ethanol+1% Cremophor+98% saline for i.p. injection.

Acute Experiment

Control rats were treated with an equal volume of the sorafenib vehicle. Sorafenib was administered once daily for 14 consecutive days. Dimiracetam, gabapentin and duloxetine were administered once daily on day 14 after sorafenib treatment.

Chronic Experiment

Control rats were treated with an equal volume of the sorafenib vehicle. Sorafenib was dissolved in a vehicle constituted by 1% ethanol+1% Cremophor+93% saline. Sorafenib was administered once daily for 14 days. Dimiracetam was administered by oral gavage twice daily for 14 days (co-administered with sorafenib).

Cold Plate Test

Rats were placed inside a stainless steel container, thermostatically set at 4° C. in a precision water-bath from KW Mechanical Workshop, Siena, Italy. Reaction times (in seconds) were measured with a stop-watch before and at regular intervals; the endpoint used was the licking of the fore or hind paws. An arbitrary cut-off time of 60 s was adopted (see Beyreuther et al., Eur. J. Pharmacol. 565, 98-104, 2007).

Statistical Analysis

All experimental results are given as the mean±S.E.M. An analysis of variance, ANOVA, followed by Fisher's protected least significant difference procedure for post hoc comparison, was used to verify significance between two means of behavioral results. Data were analyzed with the StatView software for the Macintosh (1992). P values≦0.05 were considered significant.

Blood Levels:

The blood levels were determined for 1600 bid dose (i.e. 3200 mg daily). The mean maximal and minimal levels over 24 hours were Cmin=136 micromolar and Cmax=536 micromolar.

Results

Sorafenib (10 mg/kg i.p.) administered once daily in rats induced an allodynic responses to thermal stimuli (Cold plate) on day 14.

The potential acute antiallodynic efficacy of dimiracetam (300 mg/kg p.o.), gabapentin (100 mg/kg p.o and duloxetine (30 mg/kg p.o.) was assessed before (pre test) and 15, 30 and 60 min after the injection. Among all tested compounds only dimiracetam exhibited a statistically significant effect on sorafenib induced allodynia 15 min after administration (see Table 2).

TABLE 2
EFFECT OF DIMIRACETAM IN COMPARISON WITH SOME REFERENCE
DRUGS ON SORAFENIB ALLODYNIA IN THE RAT COLD PLATE TEST

| | Licking latency (sec) | | | | |
|---|---|---|---|---|---|
| | Pre test | | | | |
| | before | 14° day | | | |
| TREATMENT | all treatments | Pre test | 15 min | 30 min | 60 min |
| VEHICLE | 27.6 ± 2.6 | 28.0 ± 3.2 | 26.4 ± 2.5 | 27.4 ± 3.4 | 27.0 ± 3.2 |
| i.p. + WATER p.o. | | | | | |
| VEHICLE i.p. | 27.2 ± 2.8 | 28.4 ± 2.1 | 29.0 ± 1.9 | 28.2 ± 2.3 | 27.2 ± 3.0 |
| DIMIRACETAM | | | | | |
| 300 mg/kg p.o. | | | | | |
| SORAFENIB | 28.6 ± 2.1 | 13.8 ± 2.0 | 14.0 ± 1.8 | 13.6 ± 1.8 | 14.4 ± 2.3 |
| 10 mg/kg i.p. + | | | | | |
| WATER p.o. | | | | | |
| DIMIRACETAM | 26.8 ± 3.0 | 15.8 ± 1.8 | 19.8 ± 1.5* | 16.0 ± 1.9 | 15.4 ± 1.4 |
| 300 mg/kg p.o. | | | | | |
| SORAFENIB | | | | | |
| 10 mg/kg i.p. | | | | | |
| GABAPENTIN | 26.8 ± 2.3 | 15.4 ± 2.0 | 13.2 ± 1.8 | 13.6 ± 1.2 | 13.8 ± 1.8 |
| 100 mg/kg p.o. | | | | | |
| SORAFENIB | | | | | |
| 10 mg/kg p.o. | | | | | |
| DULOXETINE | 27.8 ± 1.7 | 13.2 ± 1.2 | 14.2 ± 1.6 | 14.6 ± 1.5 | 13.4 ± 1.7 |
| 30 mg/kg | | | | | |
| p.o. SORAFENIB | | | | | |
| 10 mg/kg p.o. | | | | | |

Sorafenib 10 mg/kg i.p. was administered once daily for 14 days.
*P < 0.01 in comparison with sorafenib/water treated group. Vehicle: 1% Ethanol + 1% Cremophor + 98% Saline. Each group was represented by 5 rats.

Short duration of action upon one single administration is typical for compounds active in reducing neuropathic pain such as dimiracetam. Activity, even if only lasting for short duration is, however, an indicator that such compounds may achieve significant and long lasting effect after continued (chronic) administration (at least for 1 to 2 weeks).

The chronic antiallodynic effect of repeated administrations of dimiracetam (150 mg/kg p.o. b.i.d) for 14 consecutive days was evaluated on day 14, by assessing the licking latency before the last compound administration or 1, 4, 8, 24, 48 and 72 h after the end of treatment. Dimiracetam exhibited a statistically significant antiallodynic effect, which lasted for up to 48 h (tab. 2), much longer than the molecule half life in rats.

TABLE 3
EFFECT OF dimiracetam ON SORAFENIB INDUCED ALLODYNIA IN THE RAT COLD PLATE TEST

| | Licking latency (s) | | | | | | | |
|---|---|---|---|---|---|---|---|---|
| | Before all | After 14 days of treatment | | | | | | |
| TREATMENT | Treatments | Pretest | 1 h | 4 h | 8 h | 24 h | 48 h | 72 h |
| WATER + | 26.5 ± 1.3 | 26.6 ± 1.5 | 26.7 ± 1.3 | 27.6 ± 2.4 | 25.5 ± 1.4 | 27.3 ± 1.9 | 26.3 ± 1.3 | 27.5 ± 0.9 |
| VEHICLE | | | | | | | | |
| WATER + | 26.9 ± 1.4 | 14.8 ± 1.3 | 14.3 ± 1.4 | 15.1 ± 1.5 | 16.0 ± 1.1 | 14.0 ± 1.1 | 14.3 ± 1.2 | 15.0 ± 1.0 |
| SORAFENIB | | | | | | | | |
| DIMIRACETAM + | 27.3 ± 1.1 | 24.3 ± 0.8* | 23.8 ± 1.1* | 23.3 ± 1.1* | 22.8 ± 1.2* | 21.9 ± 1.1* | 19.0 ± 0.6 \hat{ } | 14.9 ± 0.6 |
| SORAFENIB | | | | | | | | |

SORAFENIB 10 mg/kg i.p. once daily for 14 days. dimiracetam 150 mg/kg p.o. b.i.d. for 14 days. Each value represents the mean of 8 rats.
\hat{ } P < 0.05;
*P < 0.01 vs Sorafenib-treated rats. Sorafenib was dissolved in a vehicle constituted by: 1% ethanol + 1% Cremophor + 98% Saline; dimiracetam was dissolved in water.

While here are shown and described presently preferred embodiments of the invention, it is to be distinctly understood that the invention is not limited thereto but may be otherwise variously embodied and practiced within the scope of the following claims.

1. Dimiracetam or a solvate thereof for the use in the treatment and/or prevention of allodynia, in particular allodynia of the hands and/or feet induced by sorafenib.

2. Dimiracetam or a solvate thereof for the use of claim 1, wherein the sorafenib is or has been administered in a combination therapy with other antitumoral chemotherapeutic principles.

3. Dimiracetam or a solvate thereof for the use of claim 1, wherein the sorafenib is administered alone, i.e. in the absence of other antitumoral chemotherapeutic principles.

4. Dimiracetam or a solvate thereof in combination with sorafenib and optionally one or more other antitumoral chemotherapeutic principles, for use in the treatment of cancer.

5. Dimiracetam or a solvate thereof for the use of claim 1, wherein dimiracetam is in the form of a mixture of its (S) and (R) enantiomers.

6. Dimiracetam or a solvate thereof for the use of claim 1, wherein dimiracetam is in the form of its isolated (S) or (R) enantiomer.

7. Dimiracetam or a solvate thereof for the use of claim 1, in a daily
oral dosage of about 1 to about 100 mg/kg, preferably about 8 to about 70 mg/kg, in particular about 15 to about 65 mg/kg, or a daily
intramuscular dosage from about 5 to about 25 mg/kg, in particular from about 8 to about 20 mg/kg, or a daily
intravenous dosage from about 2 to about 20 mg/kg, in particular 5 to 20 mg/kg, or a daily
rectal dosage from about 8 to about 70 mg/kg, preferably 10 to 65 mg/kg, in particular 15 to 35 mg/kg.

8. Dimiracetam or a solvate thereof for the use of claim 1, in a daily dosage inducing haematic levels of 100-500 micromolar.

9. Dimiracetam or a solvate thereof for the use of claim 1, in the form of a medicament suitable to administer a daily pro-Kg amount of dimiracetam of as follow:
orally at about 1 to about 100 mg/kg, preferably about 8 to about 70 mg/kg, in particular about 15 to about 65 mg/kg, or
intramuscularly at about 5 to about 25 mg/kg, in particular from about 8 to about 20 mg/kg, or
intravenously at about 2 to about 20 mg/kg, in particular 5 to 20 mg/kg and/or a daily, or
rectally at about 8 to about 70 mg/kg, preferably 10 to 65 mg/kg, in particular 15 to 35 mg/kg.

10. A pharmaceutical composition comprising dimiracetam or a solvate thereof as a first active principle and sorafenib or sorafenib and one or more other antitumor chemotherapeutic drugs as a second active principle and at least one pharmaceutically acceptable carrier either in the same administration form, or dimiracetam in a first administration form and sorafenib or sorafenib and one or more other antitumor chemotherapeutic drugs in a second administration form, wherein the first and second administration form may be the same except for the different active principles or the first and the second administration form may be different.

11. The pharmaceutical composition of claim 10, wherein the second active principle is sorafenib alone.

12. The pharmaceutical composition of claim 10, wherein dimiracetam or a solvate thereof is present in an amount for daily administration of dimiracetam of
50 to 5000 mg, preferably 400 to 3500 mg, more preferred 800 to 3200 mg in case of oral compositions;
250 to 1200 mg, preferably from 400 to 1000 mg in case of intramuscular compositions;
100 to 1000 mg, preferably from 400 to 1000 mg in case of intravenous compositions; or
400 to 3500 mg, preferably at most 3200 mg, and most preferred from 800 to 1600 mg for rectal suppository compositions (suppositories).

13. The pharmaceutical composition of claim 10, wherein dimiracetam is present in the unsolvated form.

14. (canceled)

15. A method for the treatment of sorafenib induced allodynia, in particular allodynia of the hands and/or feet, comprising the administration of an effective amount of dimiracetam.

16. (canceled)

17. The method of claim 15 wherein the sorafenib causing the allodynia is administered alone, i.e. in the absence of other antitumoral chemotherapeutic principles.

18. The method of claim 15 wherein the dimiracetam or a solvate thereof is administered in combination with sorafenib and optionally one or more other antitumoral chemotherapeutic principles in the treatment of cancer.

19. The method of claim 15 wherein dimiracetam or a solvate thereof is administered in the form of a mixture of (S) and (R) enantiomer.

20. (canceled)

21. The method of claim 15 wherein dimiracetam or a solvate thereof is administered, in a daily
oral dosage of about 1 to about 100 mg/kg, preferably about 8 to about 70 mg/kg, in particular about 15 to about 65 mg/kg, or a daily
intramuscular dosage from about 5 to about 25 mg/kg, in particular from about 8 to about 20 mg/kg or a daily
intravenous dosage from about 2 to about 20 mg/kg, in particular 5 to 20 mg/kg or a daily
rectal dosage from about 8 to about 70 mg/kg, preferably 10 to 65 mg/kg, in particular 15 to 35 mg/kg.

22. The method of claim 15 wherein dimiracetam or a solvate thereof is administered in a daily dosage inducing haematic levels of 100-500 micro-molar.

23. (canceled)

24. (canceled)

25. (canceled)

26. The method of claim 15, wherein dimiracetam is administered in the unsolvated form.

27. (canceled)
